(19) United States
(12) Patent Application Publication
(10) Pub. No.: US 2017/0042463 A1
(43) Pub. Date: Feb. 16, 2017

(54) Human Emotion Assessment Based on Physiological Data Using Semiotic Analysis

(71) Applicants: Abishek Rajkumar, Cupertino, CA (US); Ashmita Rajkumar, Cupertino, CA (US)

(72) Inventors: Abishek Rajkumar, Cupertino, CA (US); Ashmita Rajkumar, Cupertino, CA (US)

(21) Appl. No.: 14/825,181

(22) Filed: Aug. 13, 2015

(51) Int. Cl. A61B 5/16; A61B 5/00; A61B 5/1455; G06F 17/10

(52) U.S. Cl. CPC A61B 5/165; G06F 17/10; A61B 5/4815; A61B 5/14552; A61B 5/4818; A61B 5/7275; A61B 5/7278

(57) ABSTRACT

This invention disclosure describes a unique method in which a physiological disorder can be analyzed in order to determine the emotional disposition of a user. The foundation for loading physiological data and generating an emotional analysis is derived from a semiotic analysis framework in which the signs, referent, and signifier are all identified and utilized in order to complete this conversion. This method uses a time based slope-clustering algorithm in order to provide a real time human emotional assessment report based on cluster frequency.

### FIELD OF TECHNOLOGY

This disclosure relates generally to a method and system in which semiotic analysis is used in order to assess human emotions through time based slope clustering of obstructive sleep apnea data.

### BACKGROUND

Long-term research has now proven a definite relationship between any form of sleep deprivation or disturbance and the behavior of the human subject. The quality of sleep the individual receives over night significantly impacts their behavior and mood the next day to the extent that it can even alter their relationships and lifestyle. Inventions in the areas of sleep disorders and behavior focus on processes and apparatuses that detect, measure, and rectify these subject matters. However, there are few technical inventions that establish a method by which human emotions can be interpreted from sleep data. There is a dire necessity for technological breakthroughs in this area as it allows humans the opportunity to realize and improve their behavior and how they function throughout the day by analyzing their quality of sleep the prior night.

### SUMMARY

Disclosed herein is a method to report the emotional trend state and quality of a user's sleep from obstructive sleep apnea sign data but is not limited to mental illness, sleep disorders, chronic pain disorders, acute pain disorders, oral diseases, and otolaryngological diseases.

In one embodiment, semiotic analysis is utilized to take physiological sign input data and produce a human emotional assessment result that encompasses the emotional state of the user. In another embodiment, the sign parameter of the physiological disorder is identified based on the unique symptoms that the physiological disorder expresses and the sign parameter is then initialized. In another embodiment, the referent formula, which is used as a means by which the sign data input can output the signifier result, is created. In another embodiment, the signifier parameter output is identified and created, as the sign parameter will lead to the signifier output framework through the referent algorithm.

In one embodiment, the sign parameter range for each sign within the physiological disorder is created. In another embodiment, the values within the sign parameter range for every sign value are spliced by the range of human emotion values possible.

In one embodiment, a Gaussian distribution is utilized to identify the range of frequently occurring sign parameter values for each human emotion value. In another embodiment, the correlation between the sign parameter value and the human emotion value is validated.

In one embodiment, a dot product is performed of the minimum and the maximum bounds for the sign parameter range for each sign parameter. In another embodiment, a statistical test of significance is used in order to statistically identify whether the dot product emotion value varies from the original human emotion value.

In one embodiment, the human emotion value is plotted over a time series model where the time represents the time of sleep of the user. In another embodiment, the slope value is computed on an hourly interval from the referent time series model plot of the human emotion values.

In one embodiment, the Euclidean distance between the slope value and the central point of the cluster within the cluster space is computed. In another embodiment, a maximum of five cluster spaces centered on the value zero shall be created. In another embodiment, the slope value will be clustered into whichever cluster presents the smallest Euclidean distance between the slope value and the central point of the cluster.

In one embodiment, the slope value frequency counts are computed within each cluster in the cluster space. In another embodiment, after all the slope values are computed, the average slope value shall be calculated. In another embodiment, the quality of sleep and the emotional trend state, which make up the signifier output, shall be created from the average slope value that was computed.

In one embodiment, the data of the saturated oxygen level and the delta of the saturated oxygen level sign parameters of obstructive sleep apnea will be collected with a pulse oximeter. In another embodiment, a polysomnography apparatus will be used to collect the frequency of sleep apnea events per hour sign parameter. In another embodiment, the human emotion computation based on the sign parameters through a semiotic analysis framework will be done through a computer processor. In another embodiment, a database will be used to store all the sign data collected with the pulse oximeter and polysomnography and will analyze the overall trend of the human emotion of the user.

In another embodiment, the semiotic analysis framework will consist of the sleep apnea analysis, the emotion vector analysis, and the human emotion analysis. In another embodiment, the sleep apnea analysis will represent the sign analysis, the emotion vector analysis will represent the referent analysis, and the human emotion analysis will represent the signifier analysis.

In another embodiment, a pulse oximeter and a polysomnography apparatus will compute the sleep apnea analysis. In another embodiment, a computer processor will compute the emotion vector analysis. In another embodiment, the computer processor will compute the human emotion analysis and its results will be displayed on a digital apparatus.

The methods disclosed herein can be achieved as own entities that are independent of the other methods stated. Thus, any of the processes stated can be accomplished by its necessary hardware component. The details of these methods can be seen in greater detail in both the drawings and the detailed description that follows.

### BRIEF DESCRIPTION OF THE DRAWINGS

The figures illustrate the embodiments but focus specifically on obstructive sleep apnea however the overall method remains the same and can be interchangeable for any physiological disorder which affect human emotion:

FIG. 1 illustrates the semiotic component relationship between the physiological disorder, the time based slope-clustering algorithm, and the emotional output.

FIG. 2 illustrates the semiotic relationship shown in FIG. 1 in greater detail and further goes on to illustrate the relationship between all the elements of the invention disclosure.

FIG. 3 illustrates the modes and apparatus necessary to complete each part of the entire physiological to emotional conversion.

FIG. 4 shows a flow relationship of the data procurement and processing method that the hardware components achieve in FIG. 3 at each step of the way.

FIG. 5 shows the cluster outputs that exist after the computations are performed and shows a cluster space relationship with inclusion of the Euclidean distance and weightage values.

The tables and descriptions in the detailed description section will further enhance the illustrations presented.

### DETAILED DESCRIPTION

A method for which physiological data can be analyzed and processed using a semiotic analysis framework in order to output the human emotional assessment has been detailed in this invention disclosure. The embodiments of this invention disclosure detail a semiotic analysis framework that identifies the unique sign values of a physiological disorder and processes those sign values in order to provide the emotional assessment utilizing the time based slope clustering algorithm. The embodiments present in this invention disclosure detail the entire process using the sleep disorder, obstructive sleep apnea. However, the nature of this technology can be expanded beyond the scope of a single physiological disease due to the versatility that semiotic analysis provides and the flexibility that the time based slope-clustering algorithm contains.

The method of loading physiological data and producing an emotional assessment involves an intricate conversion process. This method begins by loading data of a tangible and physical sensation and applying a conversion technique to output emotional data, which is significantly dependent on the user's internal sensations and their own environment and disposition. For the input mode, a biometric apparatus that possesses the ability to measure the sign parameter is required in order to attain the physiological data. A computer device is required to compute the conversion and clustering of the physiological data into an emotional cluster space value. This human emotional assessment in turn can be outputted on any digital display model including but not limited to a computer monitor, tablet, or a cellular apparatus.

Semiotic analysis provides the groundwork and is a critical element for this entire invention disclosure. Semiotic analysis is a method of analyzing any object possible, which makes it ideal for its adaptability beyond a single element. Semiotic analysis provides a means by which an object can be signified by understanding what distinguishes it from all else. The components that highlight the object in question are labeled as the signs, which together give the object its definition and peculiarity. Identification of the signs of any object allows the user to extract that information and use it in order to understand the true significance behind an object.

The usage of semiotic analysis with physiological data is highly beneficial due to the complexity and myriad of physiological disorders known to the human body. Semiotic analysis allows for the identification and separation of all physiological disorders by locating what defines the diagnosis of the specific disorder as a sign.

Obstructive sleep apnea is a sleep disorder in which oxygen flow is compromised due to the muscles in the body's air pathway collapsing and inhibiting oxygen flow. Obstructive sleep apnea is one of many sleep disorders known to mankind but contains a few signs that distinguish it from other physiological disorders. The following three tables respectively show sign values of obstructive sleep apnea and their correlation to a human emotional value.

TABLE 1
This table presents the conversion values and Gaussian
statistics between one of the signs, the saturated
oxygen level, and the human emotion scale.

| Emotion | Saturated Oxygen | | |
|---|---|---|---|
| Value | Range (%) | μ (%) | σ (%) |
| −5★ | 0-80 | 79 | 0.5 |
| −4 | 80-81.6 | 81.2 | 0.15 |
| −3 | 81.6-83.3 | 82.7 | 0.2 |
| −2 | 83.3-85 | 84.4 | 0.2 |
| −1 | 85-86.6 | 86 | 0.225 |
| 0 | 86.6-88.3 | 87.6 | 0.3 |
| 1 | 88.3-90 | 89.2 | 0.3 |
| 2 | 90-91.6 | 90.9 | 0.3 |
| 3 | 91.6-93.3 | 92.5 | 0.25 |
| 4 | 93.3-95 | 94.1 | 0.225 |
| 5 [symbol] | 95-100 | 97.1 | 0.85 |

TABLE 2
This table presents the conversion values and Gaussian statistics
between one of the signs, the frequency of obstructive sleep apnea
events for every one hour interval, and the human emotion scale.

| Emotion | Frequency of Obstructive | | |
|---|---|---|---|
| Value | Sleep Apnea Events per Hour | μ | σ |
| −5★ | 28-60 | 28.5 | 0.4 |
| −4 | 25-27 | 26.4 | 0.175 |
| −3 | 22-24 | 22.3 | 0.2 |
| −2 | 19-21 | 20.3 | 0.2 |
| −1 | 16-18 | 17.2 | 0.2 |
| 0 | 13-15 | 14.2 | 0.225 |
| 1 | 10-12 | 11.1 | 0.25 |
| 2 | 7-9 | 8 | 0.3 |
| 3 | 4-6 | 5.1 | 0.3 |
| 4 | 2-3 | 2.6 | 0.2 |
| 5 | 0-1 | 0.7 | 0.15 |

TABLE 3
This table presents the conversion values and Gaussian statistics
between the final sign, the absolute instantaneous delta value
of the saturated oxygen level, and the human emotion scale.

| Emotion | \|Δ\| of Saturated | | |
|---|---|---|---|
| Value | Oxygen Level | \|μ\| | σ |
| −5★ | 20-100 | 20.3 | 0.5 |
| −4 | 18-19 | 18.1 | 0.2 |
| −3 | 16-17 | 16.3 | 0.2 |
| −2 | 14-15 | 14.4 | 0.225 |
| −1 | 12-13 | 12.4 | 0.25 |
| 0 | 10-11 | 10.5 | 0.225 |
| 1 | 8-9 | 8.4 | 0.3 |
| 2 | 6-7 | 6.5 | 0.325 |
| 3 | 4-5 | 4.5 | 0.3 |
| 4 | 2-3 | 2.6 | 0.25 |
| 5 | 0-1 | 0.7 | 0.225 |

For each of the sign values in tables 1 through 3, the human emotion value of −5 presents a unique sign parameter range compared to the rest of the human emotion values. The −5 human emotion value encompasses not only the minimum plausible sign values that a human will most likely fall under but it also contains the physical minimum value possible. The discrepancy between the physical minimum sign value and the minimum feasible sign value causes the range of the −5 human emotion sign parameter to be larger than the other human emotion values. Although the sign parameter range is significantly larger than the rest of the ranges, the population mean is still extremely close the maximum bound in table 1 and the minimum bound in tables 2 and 3. This proximity to a minimum or maximum bound creates a significant skewness. The reason for this proximity to a bound of the range is because even though the range is huge, most of the data points that will fall in the −5 human emotion category will barely miss the cutoff for the −4 human emotion range. Occasionally, there will be outliers to the −5 human emotion range which causes the population standard deviation to be larger compared to the rest of the human emotion range's population standard deviations.

[symbol] The +5 human emotion range value in table 1 also displays unique values due to the physiological nature of the human body at that sign parameter range. The human body's saturated oxygen level is constantly oscillating and the medical field considers that a saturated oxygen level of 95% or above is considered excellent and that there is no significant difference between saturated oxygen percentage values. The sign parameter range of the +5 human emotion is slightly larger than all the intermediate human emotion values however since all the values within the range are often reached by the human body, the population mean is relatively centered around this range. Since the sign range is larger yet all the values within the range are covered unlike the −5 human emotion range with its extreme skewness, the population standard deviation is also slightly larger to account for the distribution of the sign values.

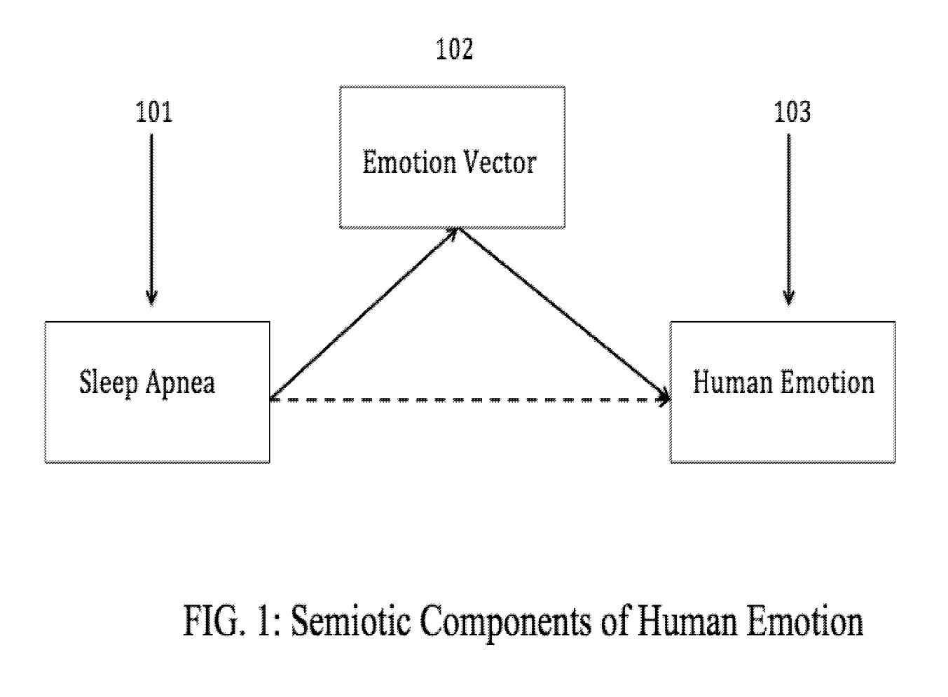

FIG. 1 shows an overall relationship between the main components of this invention disclosure and their semiotic relationship. The three components of any semiotic relationship include the signs, referent, and the signifier. The signs create unique identification points that allow for the defining of the signifier. The sleep apnea, which serves as, a physiological disorder for the invention disclosure method serves as the sign in this semiotic analysis frame and is component 101. The time based slope-clustering algorithm serves as the referent by which the signifier, component 103, can be derived from the sign input and is component 102. The human emotion assessment is the signifier in this semiotic analysis relationship as it is the desired output that needs to be determined and is represented by component 103. The signs amalgamated together lead towards the signifier as illustrated by the arrow from component 101 to component 103. Since the signs and the signifier are each its own entity, it is burdensome to establish a direct relationship between the two semiotic branches. Thus, the referent serves as a means by which the sign can lead to the signifier. The signs serve as a form of input in which the referent can aggregate this data and output the signifier. This relationship between the sign, referent, and signifier can be seen in a two-step process. The first part of this relationship is the sign to the referent where the signs serve as input modes as illustrated by the arrow between component 101 to component 102. The referent then takes the sign data and computes an output value that ends up forming the signifier as shown in the relationship between component 102 to component 103. The relationship between component 102 and component 103 makes up the second part of the relationship between the sign, referent, and the signifier.

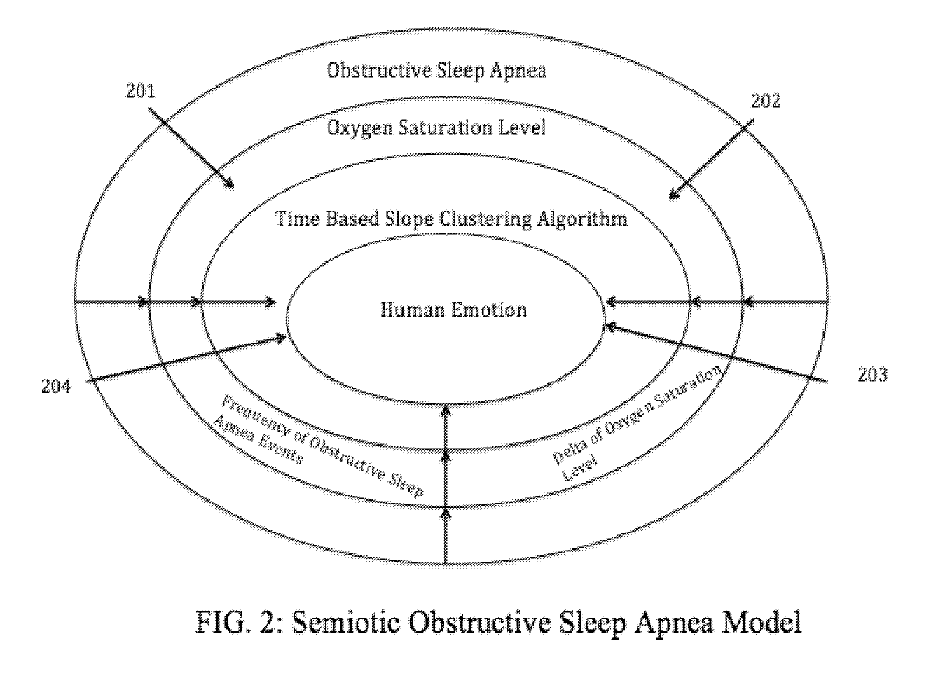

FIG. 2 builds upon the semiotic analysis relationship in FIG. 1 by going beyond just the semiotic relationship on its own and showing the space relationship between the components of the semiotic method. Space 201 is the starting point for the entire semiotic analysis framework and encompasses the largest and the entry portion of the figure as it represents the physiological disorder that will be highlighted on, in this case, obstructive sleep apnea. Space 202 lies within space 201 as space 202 contains the three signs that have been identified as unique to obstructive sleep apnea and have been extracted from the physiological disorder. Information on each of the three signs and their association with the human emotion spectrum can be seen in tables 1 through 3. Space 203 contains the referent of the semiotic analysis method with the time based slope clustering algorithm that takes the signs in space 202 and produces the signifier which can be found in the final space 204 which contains the human emotion assessment output. FIG. 2. further demonstrates an important segmented and sequential relationship between the components of the semiotic analysis structure by the arrows which start off at space 201 and in sequential spacing order then go into the sign space 202, then the referent space 203, and finally signifier space 204.

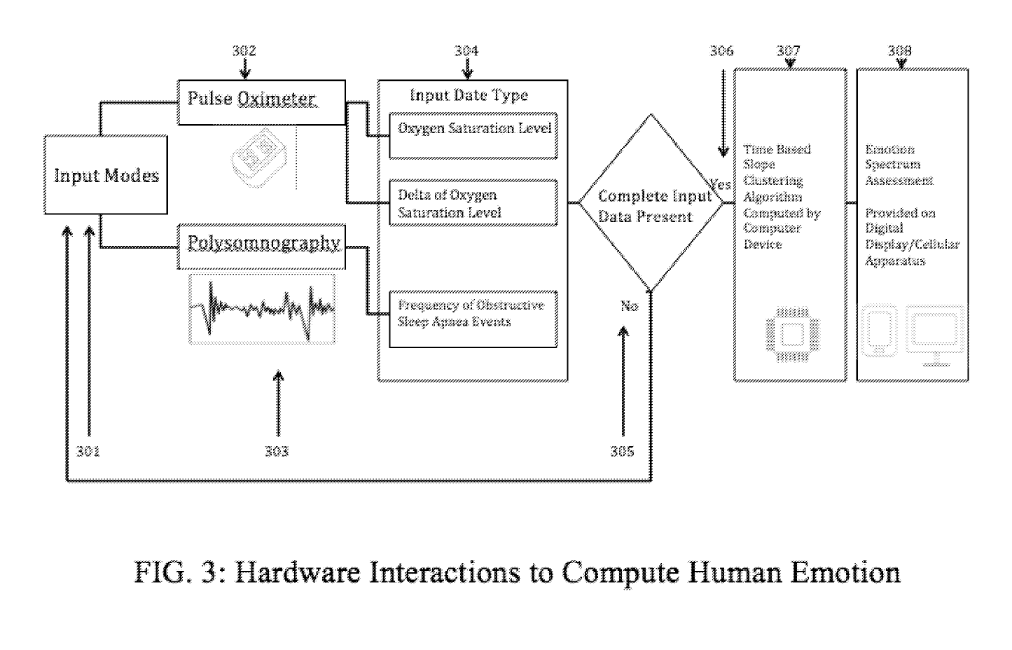

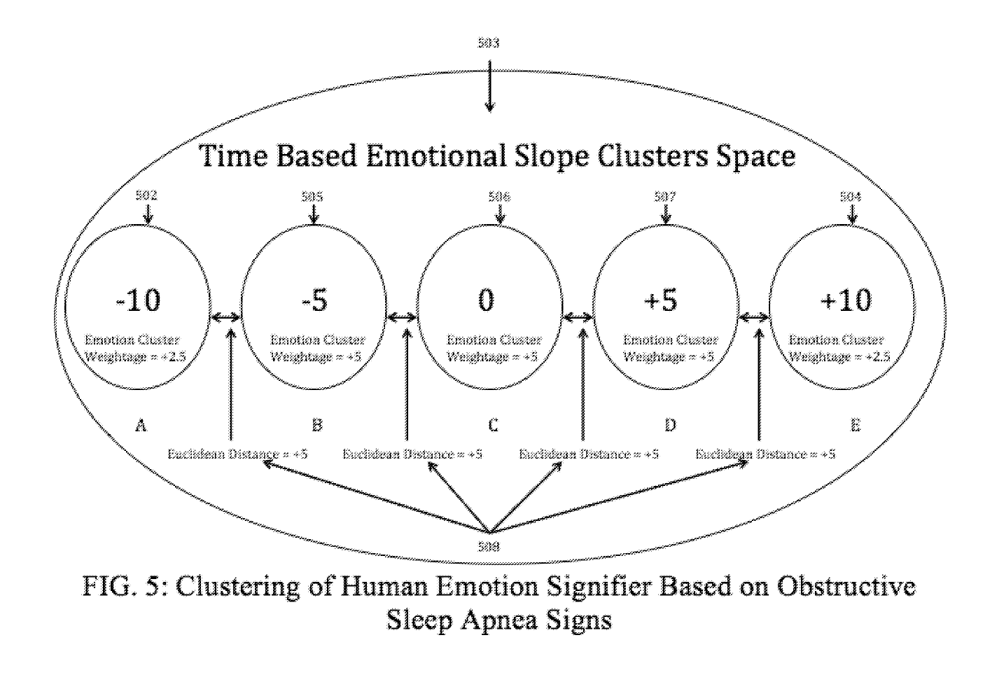

FIG. 3 shows a flowchart of the computational process that focuses on the apparatuses necessary at each stage in the process. The primary step is collecting the input mode physiological data in 301. For obstructive sleep apnea, a pulse oximeter, 302, and a polysomnography machine, 303, were necessary to collect the three sign data types that form the input modes in 301. However, the medical machinery necessary varies by physiological disorders as each disorder carries unique signs that have their own means and tools by which the data can be collected. Component 304 shows the input data variables that the pulse oximeter and the polysomnography machines collect where the oximeter collects the saturated oxygen level and the delta of the saturated oxygen level while the polysomnography machine records the frequency of obstructive sleep apnea events on an hourly interval. Components 305 and 306 together present a conditional statement in the process that validates whether all the input data has been collected. If option 305 is selected, the data input portion will be iterated until the full dataset has been received which prompts option 306 to activate and moves to the next step of the process, 307. Component 307 is the time based slope clustering algorithm which is performed by the computer device since the input data types in 304 contain large sets of numerical values that must be processed by the algorithm in order to get the emotional signifier assessment. Component 307 which is also the referent in the semiotic relationship will output the signifier, the human emotion assessment, once the computer device is done analyzing through all the data as seen in component 308. This assessment in 308 will be presented on a digital, cellular, or tablet display system. In terms of the assessment report, each time the data processing loop goes through the time based slope-clustering algorithm, a discrete slope value will be computed and placed into a cluster space. After all the slope values have been calculated and sorted, the assessment will provide a detailed cluster frequency of the amount of times a user's sign values indicated that their sleep fell into a particular cluster space. Using this frequency, an overall statement of the quality of sleep and what the user's emotional trend state will be provided in real time. Using the frequency values, an average will be calculated of all the frequency counts of the slope clusters and this average will then be used to determine the overall statement about the user's sleep quality and emotional trend direction. The average can fall within the range of positive 10 to −10 which covers the entire Euclidean distance of the slope cluster space as seen in component 503 in FIG. 5.

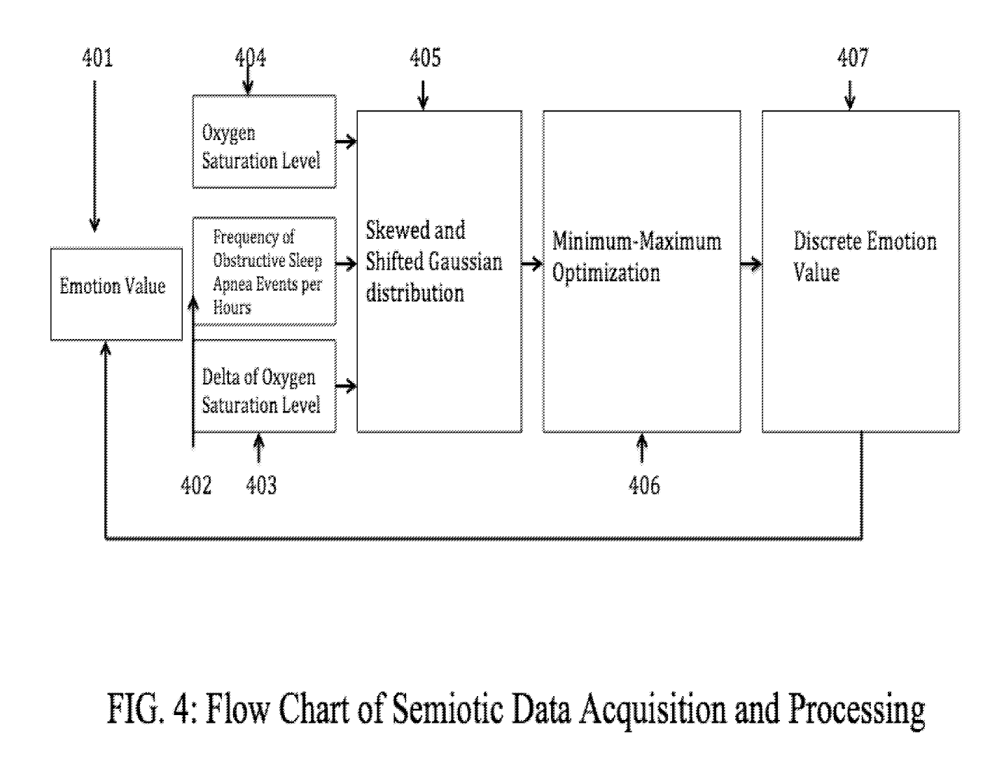

FIG. 4 approaches the same concepts displayed in FIG. 3 but rather than focusing on the hardware aspects of the invention disclosure, FIG. 4 focuses on the math behind the time based slope clustering algorithm. The starting point, 401, begins with the emotional value that corresponds the values of the three signs, which can be found in tables 1 through 3. Afterwards, this emotional value needs to be validated for accuracy by comparing it with all three signs values in components 402, 403, and 404. The signs values create a correspondence to the emotional value however a computation applied to all three sign values together prove that the human emotional value is approved. In tables 1 through 3, the sign variable for each human emotional value is given a range. This range was found by utilizing a Gaussian distribution model, 405, that could statistically deduct the range of values within each sign parameter per human emotion that were most likely going to occur. Using both the minimum and the maximum points for each human emotional value allows the user to find the range of human emotional values that are statistically significant. By multiplying the corresponding minimum and maximum values for each sign together provides a minimum and maximum emotional value. If the range of minimum and maximum emotional values contains the starting emotional value 401, then the 401 value has been validated as accurate which presents the minimum and maximum optimization method seen in 406. Once this human emotion value has been verified, it will be plotted on a time series model for all the emotion value points collected for the user's sleep during that night. Using a slope clustering method, the slope values will be calculated from the time series model and clustered into an emotion spectrum as seen in step 407.

The pseudo code for the entire invention disclosure process is as follows:
Input sign variables: M(O₂)—saturated oxygen level, M(OSA_{E})—frequency of apnea events per hour, M(D)—change in saturated oxygen level
Compute emotion value: f(E)=M(O₂)ₜ*M(OSA_{E})ₜ*M(D)ₜ
Identify sign based on f(E)
Compute slope from f(E) for each hour for range of n hours where n is the number of hours the user has slept
Cluster categories and characteristics of emotion trends must be defined with a maximum of 5 clusters
Find the Euclidean distance between slope values and cluster categories
Place slope value in the cluster category where slope value has the minimum Euclidean distance from the category
Place all slope points in each cluster till all points have been computed and clustered
Cluster analysis will be computed and a human emotional assessment will be presented from the cluster analysis of slope data points

FIG. 5 shows a detailed representation of the cluster space, 503, and categories where each of the slope data points for a single time must be clustered into. There is a maximum of 5 clusters in which all the data points can be sorted into which are the −10 (502), −5 (505), 0 (506), +5 (507), and +10 (504) clusters. The values that represent each cluster are also the central points of their clusters. Between any individual two clusters is a Euclidean Distance, (508), which is representative of the distance between the central points of those two respective clusters. For each of the five clusters in this diagram, the Euclidean distance between any two adjacent clusters stays constant at a value of 5. As the Euclidean distance between any slope value and a central point of a cluster approaches 2.5, the slope value will lose its cluster strength and will be placed in the next cluster depending on which direction it is approaching the Euclidean distance value of 2.5. Though the distance between the clusters is constant, the cluster weightage, 501, is not as the range of values within a single cluster varies by cluster. Both the 502 and the 504 clusters have a cluster weightage of only 2.5 as they both are the extreme ends of the emotion spectrum and thus, values beyond their minimum and maximum occur at such an insignificant rate. The clusters between 502 and 504 consisting of the 505, 506, and 507 clusters all have an equal cluster weightage of 5. Note that the cluster weightage difference between clusters can be distinguished by the size of the circle encapsulating the individual clusters.

### INDUSTRIAL APPLICATION

This invention can construct an emotional analysis report of a user by evaluating physiological disorder data of the same user via a semiotic mapping analysis. This technology can best serve and be utilized by the hospital and medical industries in which it is critical to understand the emotions that are ongoing within a patient in order to allow the medical care providers to administer the best care possible.

This invention disclosure serves as a pioneer technology towards semiotic analysis in the healthcare sector. Semiotic analysis has tremendous potential in terms of optimizing the physical examination portion of the health care interaction between a physician and the patient. By identifying the sign, referent, and signifier of the physical examination portion of the healthcare visit; healthcare administrators can recognize areas of weaknesses and work towards improving patient satisfaction. In terms of the physical examination that a physician provides to the patients, the sign would be the symptoms that the patient expresses. The referent would be the diagnosis and medicine prescribed by the physician and the signifier would be the patient eventually being cured and having both a better lifestyle and health.

What is claimed is:

1. A method, comprising:
Performing a semiotic analysis on physiological data in order to produce an emotional output that signifies the emotional sensations of the human body;
Identifying and initializing sign parameter values that embody and define the symptoms that a physiological disorder displays;
Creating a referent formula that serves as an algorithm by which the signifier value can ultimately be derived from the sign parameters values;
Identifying a signifier output by which the sign parameter value can serve as data that will ultimately lead to a conclusion about the signifier framework.

2. The method of claim 1, further comprising:
Creating the sign parameter range of the physiological disorder;
Splicing the values within the sign parameter range of the physiological disorder by human emotion value.

3. The method of claim 1, wherein a Gaussian distribution is applied in order to find the range of the highest occurring sign parameter values per emotion value.

4. The method of claim 1, wherein the sign parameter value is validated with the human emotion values correlation value.

5. The method of claim 4, further comprising:
Performing a dot product of all the minimum and maximum bounds of all sign parameter ranges for each sign parameter;
Performing a statistical test of significance to see if the dot product emotion value has a statistical difference from the original human emotion value.

6. The method of claim 5, wherein the human emotion value shall be plotted on a time series model where time represents the duration of the user's sleep.

7. The method of claim 6, wherein a slope value is computed on a one-hour interval from the referent algorithm plot.

8. The method of claim 1, wherein the Euclidean distance between said slope value and the central cluster points within a cluster space.

9. The method of claim 8, wherein said cluster space shall contain a maximum of five clusters.

10. The method of claim 8, wherein the slope value shall be clustered into the cluster in which the Euclidean distance is at its minimum value.

11. The method of claim 1, wherein the frequencies of the slope value counts within each cluster in the cluster space are computed.

12. The method of claim 11, wherein the average slope value of all the slope values recorded during the user's sleep duration is computed.

13. The method of claim 1, wherein the emotional trend state and sleep quality of the user is created from the average slope value that was computed.

14. A system, comprising:
A pulse oximeter to collect both the saturated oxygen level and the change in saturated oxygen level sign parameters;
A polysomnography apparatus to collect the frequency of sleep apnea events per hour sign parameter;
A processor to house the human emotion computation based on sign parameters which form the basis of semiotic analysis;
A database to store the historical data and analyze the overall trend of the human emotional state.

15. The system of claim 14, wherein the semiotic analysis consists of the sleep apnea analysis, the emotion vector analysis, and the human emotion analysis.

16. The system of claim 15, wherein the sleep apnea analysis, the emotion vector analysis, and the human emotion analysis respectively symbolize the sign analysis, referent analysis, and the signifier analysis.

17. The system of claim 14, wherein the sleep apnea analysis will be computed using both the pulse oximeter and the polysomnography apparatus.

18. The system of claim 14, wherein the emotion vector analysis will be computed by a computer processor.

19. The system of claim 14, wherein the human emotion analysis will be computed by a computer processor and the output shall be displayed on a digital apparatus.
